(19) United States
(12) Patent Application Publication
(10) Pub. No.: US 2007/0065365 A1
(43) Pub. Date: Mar. 22, 2007

(54) Abuse-resistant transdermal system

(75) Inventors: Heinrich Kugelmann, Aachen (DE); Johannes Bartholomaeus, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/583,808

(22) Filed: Oct. 20, 2006

Related U.S. Application Data
(63) Continuation of application No. PCT/EP05/04280, filed on Apr. 21, 2005.

(30) Foreign Application Priority Data
Apr. 21, 2004 (DE) 10 2004 019 916.7

(51) Int. Cl. A61K 49/00; A61K 9/70

(52) U.S. Cl. 424/10.1; 424/449

(57) ABSTRACT

An abuse-resistant transdermal system which contains, in addition to one or more active ingredients with potential for abuse, at least one gel-forming agent in quantities such that it forms a gel with a minimum quantity of an aqueous liquid, and contains as further agents which complicate or prevent abuse at least one emetic, and/or at least one dye as an aversive agent.

### CROSS REFERENCE TO RELATED APPLICATIONS

This application is a continuation of international application no. PCT/EP2005/004280, filed Apr. 21, 2005, designating the United States of America and published in German on Nov. 3, 2005 as WO 2005/102294, the entire disclosure of which is incorporated herein by reference. Priority is claimed based on Federal Republic of Germany patent application no. DE 10 2004 019 916.7, filed Apr. 21, 2004.

### FIELD OF THE INVENTION

The present invention relates to an abuse-resistant transdermal system which contains, apart from one or more active ingredients with potential for abuse, at least one gel-forming agent in quantities such that it forms a gel with a minimum quantity of an aqueous liquid, and comprises as further agents which complicate or prevent abuse
at least one emetic, (c) and/or
at least one dye (d) as an aversive agent and
optionally at least one irritant (a) and/or
optionally at least one antagonist (b) for the active ingredient(s) with potential for abuse.

### BACKGROUND OF THE INVENTION

Many pharmaceutical active ingredients, in addition to having excellent activity in their appropriate application, also have potential for abuse, i.e. they can be used by an abuser to bring about effects other than those intended. Opioids, for example, which are highly active in combating severe to very severe pain, are frequently used by abusers to induce a state of narcosis or euphoria.

In order to make abuse possible, for example oral dosage forms, such as tablets or capsules are comminuted, inter alia ground in a mortar, by the abuser, the active ingredient is extracted from the resultant powder using a preferably aqueous liquid and the resultant solution, optionally after being filtered through cotton wool or cellulose wadding, is administered parenterally, in particular intravenously. An additional phenomenon of this kind of administration, in comparison with abusive oral administration, is a further accelerated increase in active ingredient levels giving the abuser the desired effect, namely the “kick” or “rush”.

U.S. Pat. No. 4,070,494 proposed adding a swellable agent to the oral or rectal dosage form in order to prevent abuse. When water is added to extract the active ingredient, this agent swells and ensures that the filtrate separated from the gel contains only a small quantity of active ingredient.

However, not only oral or rectal dosage forms comprising active ingredients which may be abused are used to achieve states similar to narcosis. Transdermal systems, such as patches, which are used to release an active ingredient into the human or animal body, are also chopped up into small pieces by an abuser, extracted using a preferably aqueous liquid and the resultant solution, optionally after being filtered through cotton wool or cellulose wadding, is administered parenterally, in particular intravenously.

In order to complicate such abuse, International application no. WO 03/013479 proposes adding an opioid antagonist and another agent which complicates abuse, such as for example a compound which forms a gel with an aqueous liquid, to the transdermal system.

Despite this measure for complicating parenteral, in particular intravenous, abuse of active ingredients with potential for abuse, a major requirement still remains to prevent any kind of abusive use in such a manner that the abuser is either discouraged as far as possible even from abusively taking the active ingredient with potential for abuse or, after abusively taking the active ingredient with potential for abuse, this active ingredient does not remain in the body sufficiently long to bring about therein the states associated with abusive intake of an active ingredient with potential for abuse.

### SUMMARY OF THE INVENTION

The foregoing object is achieved in accordance with the present invention by providing an abuse-resistant transdermal system which contains, in addition to one or more active ingredients with potential for abuse, at least one gel-forming agent in quantities such that it forms a gel with a minimum quantity of an aqueous liquid, and comprises as a further agent which complicates or prevents abuse
at least one emetic, (c) and/or
at least one dye (d) as an aversive agent and
optionally at least one irritant (a) and/or
optionally at least one antagonist (b) for the active ingredient(s) with potential for abuse.

Active ingredients with potential for abuse, preferably pharmaceutical active ingredients with potential for abuse, are, like the dosage or production processes thereof, known to the person skilled in the art and may be present in the transdermal system according to the invention as such, in the form of the derivatives thereof, in particular esters, amides or ethers, or in each case in the form of the transdermally administrable physiologically acceptable compounds thereof, preferably in the form of the salts thereof, very particularly preferably as hydrochlorides, or solvates.

The transdermal system according to the invention is also suitable for the administration of two or more active ingredients with potential for abuse. The transdermal system preferably comprises only one active ingredient with potential for abuse for transdermal administration.

The transdermal system according to the invention is preferably suitable for preventing the abuse of at least one transdermally administrable pharmaceutical active ingredient with potential for abuse which is selected from the group comprising narcotic analgesics, opioids, tranquilizers, preferably benzodiazepine, stimulants and further narcotics.

The transdermal system according to the invention is very particularly suitable for preventing the abuse of at least one transdermally administrable opioid, tranquilizer or another narcotic, which is selected from the group comprising N-{1-[2-(4-ethyl-5-oxo-2-tetrazolyl-1-yl)ethyl]4-methoxymethyl-4-piperidyl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methylphenethylamine(amphetamine), 2-(α-methylphenethylamino)-2-phenylacetonitrile (amphetaminil), 5-ethyl-5-isopentylbarbituric acid (amobarbital), anileridine, apocodeine, 5,5-diethylbarbituric acid (barbital), bemidone, benzylmorphine, bezitramide, 7-bromo-5-(2-pyridyl)-1H-1,4-benzodiazepine-2(3H)-one (bromazepam), 2-bromo-4-(2-chlorophenyl)-9-methyl-6H-thieno[3,2-f][1,2,4]triazolo[4,3-a][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepin-3-yl) dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine 4-oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, carfentanil, clofedanol, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate] (cocaine), 4,5α-epoxy-3-methoxy-17-methyl-7-morphinan-6α-ol (codeine), 5-(1-cyclohexenyl)-5-ethyl barbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl)propionate (dextropropoxyphene), dextromethorphan, dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), ephedrine, pseudoephedrine, eptazocine-, 8-chloro-6-phenyl-4H-[1,2,4]triazolo[4,3-(a)][1,4]benzodiazepine (estazolam), ethoheptazine, ethylmethylthiambutene, ethyl[7-chloro-5-(2-fluorophenyl)-2,3-dihydro-2-oxo-1H-1,4-benzodiazepine-3-carboxylate](ethyl loflazepate), 4,5α-epoxy-3-ethoxy-17-methyl-7-morphinen-6α-ol (ethyl morphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine(fencamfamine), 7-[2-α-methylphenethylamino)ethyl]-theophylline) (fenethylline), 3-(α-methylphenethyl-amino)propionitrile (fenproporex), fenpipramide, N-(1-phenethyl-4-piperidyl)propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 11-chloro-8,12b-dihydro-2,8-dimethyl-12b-phenyl-4H-[1,3]oxazino[3,2-d][1,4]benzodiazepine-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 6-(2-chlorophenyl)-2-(4-methyl-1-piperazinylmethylene)-8-nitro-2H-imidazo[1,2-a][1,4]-benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethyl-amine (methamphetamine), (±)-6-dimethylamino-4,4-diphenol-3-heptanone (methadone), 2-methyl-3-o-tolyl-4(3H)-quinazolinone (methaqualone), methyl[2-phenyl-2-(2-piperidyl)acetate](methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5a][1,4]benzodiazepine (midazolam), 2-(benzhydrylsulfinyl)acetamide(modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6Hα)-one (nabilone), nalbuphene, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2(3H)-one (nordazepam), norlevorphanol, 6-dimethylamino-4,4-diphenyl-3-hexanone (normethadone), normorphine, norpipanone, the exudation from plants belonging to the species Papaver somniferum (opium), 7-chloro-3-hydroxy-5-phenyl-1H-1,4-benzodiazepin-2(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papaveretum, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl-(1-methyl-4-phenyl-4-piperidinecarboxylate) (pethidine), phenadoxone, phenomorphane, phenazocine, phenoperidine, piminodine, pholcodine, 3-methyl-2-phenylmorpholine (phenmetrazine), 5-ethyl-5-phenylbarbituric acid (phenobarbital), α,α-dimethyl-phenethylamine (phentermine), 7-chloro-5-phenyl-1-(2-propynyl)-1H-1,4-benzodiazepin-2(3H)-one (pinazepam), α-(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate}(remifentanil), 5-sec-butyl-5-ethylbarbituric acid (secbutabarbital), 5-allyl-5-(1-methylbutyl)-barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepin-2(3H)-one (tetrazepam), ethyl (2-dimethylamino-1-phenyl-3-cyclohexene-1-carboxylate) (tilidine (cis and trans)), tramadol, 8-chloro-6-(2-chlorophenyl)-1-methyl-4H-[1,2,4]triazolo-[4,3-a][1,4]benzodiazepine (triazolam), 5-(1-methylbutyl)-5-vinylbarbituric acid (vinylbital), (1R,2R)-3-(3-dimethylamino-1-ethyl-2-methyl-propyl)phenol, (1R,2R,4S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1RS,3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl 2-(4-isobutoxy-phenyl)-propionate, 3-(2-dimethylaminomethyl-1-hydroxy-cyclohexyl)phenyl 2-(6-methoxy-naphthalen-2-yl)-propionate, 3-(2-dimethylaminomethyl-cyclohex-1-enyl)-phenyl 2-(4-isobutyl-phenyl)-propionate, 3-(2-dimethylaminomethyl-cyclohex-1-enyl)-phenyl 2-(6-methoxy-naphthalen-2-yl)-propionate, (RR-SS)-2-acetoxy-4-trifluoromethyl-benzoic acid 3-(2-dimethylamino-methyl-1-hydroxy-cyclohexyl)-phenyl ester, (RR—SS)-2-hydroxy-4-trifluoromethyl-benzoic acid 3-(2-dimethylaminomethyl-1-hydroxy-cyclohexyl)-phenyl ester, (RR-SS)-4-chloro-2-hydroxy-benzoic acid 3-(2-dimethylaminomethyl-1-hydroxy-cyclohexyl)-phenyl ester, (RR-SS)-2-hydroxy-4-methyl-benzoic acid 3-(2-dimethylaminomethyl-1-hydroxy-cyclohexyl)-phenyl ester, (RR—SS)-2-hydroxy-4-methoxy-benzoic acid 3-(2-dimethylaminomethyl-1-hydroxy-cyclohexyl)-phenyl ester, (RR-SS)-2-hydroxy-5-nitro-benzoic acid 3-(2-dimethylaminomethyl-1-hydroxy-cyclohexyl)-phenyl ester, (RR-SS)-2′,4′-difluoro-3-hydroxy-biphenyl-4-carboxylic acid 3-(2-dimethylaminomethyl-1-hydroxy-cyclohexyl)-phenyl ester together with corresponding stereoisomeric compounds, in each case the corresponding derivatives thereof, in particular amides, esters or ethers, and in each case the physiologically acceptable compounds thereof, in particular the salts and solvates thereof, particularly preferably hydrochlorides.

The transdermal system according to the invention is in particular suitable for preventing abuse of an opioid active ingredient selected from among the group comprising (1R,2R)-3-(3-dimethylamino-1-ethyl-2-methyl-propyl)phenol, (2R,3R)-1-dimethylamino-3-(3-methoxyphenyl)-2-methyl-pentan-3-ol, (1RS,3RS,6RS)-6-dimethylaminomethyl-1-(3-methoxyphenyl)-cyclohexane-1,3-diol, (1R,2R)-3-(2-dimethylaminonethyl-cyclohexyl)phenol, the physiologically acceptable salts thereof, preferably hydrochlorides, physiologically acceptable enantiomers, stereoisomers, diastereomers and racemates and the physiologically acceptable derivatives thereof, preferably ethers, esters or amides.

These compounds and the process for the production thereof are described in U.S. Pat. No. 6,248,737 (=EP 693,475) and U.S. Pat. No. 5,801,201 (=EP 780,369), respectively, which are hereby incorporated by reference and are deemed to be part of the disclosure.

The transdermal system according to the invention is very particularly preferably suitable for preventing the abuse of opioids, preferably analgesically active opioids, particularly preferably of at least one opioid selected from among the group comprising morphine, oxycodone, buprenorphine, sulfentanil, hydromorphone, carfentanil, lofentanyl and fentanyl, particularly preferably buprenorphine, the derivatives thereof, such as esters, ethers or amides, or in each case the physiologically acceptable compounds thereof, preferably the salts thereof, such as hydrochlorides or sulfates, or solvates, in each case the stereoisomeric compounds, enantiomers, diastereomers and/or racemates thereof.

Provision of the transdermal system according to the invention ensures that an abuser is discouraged from any kind of abuse, i.e. not only from parenteral, in particular intravenous abuse, but also from oral or nasal abuse, or, if the active ingredient is nevertheless taken abusively, the active ingredient does not remain in the abuser's body long enough to bring about the effects caused by abuse, in particular the kick or rush.

To this end, the transdermal system according to the invention comprises not only at least one gel-forming agent in quantities such that it forms a gel with a minimum quantity of an aqueous liquid, but also at least one physiologically acceptable dye (d) which is soluble in an aqueous solution and/or at least one emetic (c) as a further compound which complicates or prevents abuse.

For the purpose of abusive use, preferably for intravenous administration, of an active ingredient with potential for abuse, an abuser conventionally attempts to extract said active ingredient from the transdermal system with the assistance of an aqueous liquid, preferably water. In the development of the transdermal system according to the invention, a gel may form directly on the transdermal system from which no appreciable quantity of active ingredient can be extracted, or the aqueous extract from the transdermal system is converted by the viscosity-increasing agent, i.e. the gel-forming agent, into a gel which can no longer be filtered or administered.

Since, in one of the preferred embodiments thereof, the transdermal system according to the invention furthermore contains as a further abuse-preventing agent a dye which is soluble in an aqueous liquid, preferably in water, when aqueous extraction of the active ingredient is attempted for the purpose of abusive use, an intense color is imparted to the gel or extract. This color acts as an additional deterrent to the potential abuser, such that the abuser is deterred not only from parenteral, preferably intravenous, administration due to the health risks associated with the administration of high-viscosity liquids, but also from oral and/or nasal administration. Suitable dyes and the quantities required for this purpose may be found in published U.S. patent application no. US 2005/089475 (=WO 03/015531), the disclosure of which is incorporated herein by reference.

As explained above, the gel-forming agent, i.e. the viscosity-increasing agent, is added to the transdermal system in such quantities that, with the assistance of a necessary minimum quantity of an aqueous liquid, preferably in the aqueous extract obtained from the transdermal system, a gel is formed which cannot be administered without risk and which preferably also remains visually distinguishable on introduction into a further quantity of an aqueous liquid.

For the purposes of the present invention visually distinguishable means that the active ingredient-containing gel formed with the assistance of a necessary minimum quantity of aqueous liquid, when introduced, preferably with the assistance of a hypodermic needle, into a further quantity of aqueous liquid at 37° C., remains substantially insoluble and cohesive and cannot straightforwardly be dispersed in such a manner that it can safely be administered parenterally, in particular intravenously. The material preferably remains visually distinguishable for at least one minute, preferably for at least 10 minutes.

The increased viscosity of the extract makes it more difficult or even impossible for it to be passed through a needle or injected. If the gel remains visually distinguishable, this means that the gel obtained on introduction into a further quantity of aqueous liquid, for example by injection into blood, initially remains in the form of a largely cohesive thread, which, while it may indeed be broken up mechanically into smaller fragments, cannot be dispersed or even dissolved in such a manner that it can safely be administered parenterally, in particular intravenously.

Intravenous administration of such a gel would most probably result in obstruction of blood vessels, associated with serious damage to the health of the abuser.

In order to verify whether a viscosity-increasing agent is suitable as a gel-forming agent for use in the dosage form according to the invention, the active ingredient is mixed with the viscosity-increasing agent and suspended in 10 m1 of water at a temperature of 25° C. If this results in the formation of a gel which fulfils the above-stated conditions, the corresponding viscosity-increasing agent is suitable for averting or preventing abuse in the transdermal system according to the invention.

One or more viscosity-increasing agents may be added to the transdermal system which are selected from the group comprising microcrystalline cellulose with 11 wt. % carboxymethylcellulose sodium (Avicel® RC 591), carboxymethylcellulose sodium (Blanose®, CMC-Na C300P®, Frimulsion BLC-5®, Tylose C300 P®), polyacrylic acid, acrylate copolymers which are preferably crosslinked, (Carbopol® 980 NF, Carbopol® 981), locust bean flour (Cesagum® LA-200, Cesagum® LID/150, Cesagum® LN-1), pectins, preferably from citrus fruit and apples (Cesapectin® HM Medium Rapid Set), sucrose acetate isobutyrate, waxy maize starch (C*Gel 04201®), sodium alginate (Frimulsion ALG (E401)®), guar flour (Frimulsion BM®, Polygum 26/1-75®), iota carrageenan (Frimulsion D021®), karaya gum, gellan gum (Kelcogel F®, Kelcogel LT100®), galactomannan (Meyprogat 150®), tara stone flour (Polygum 43/1®), propylene glycol alginate (Protanal-Ester SD-LB®), sodium hyaluronate, tragacanth, tara gum (Vidogum SP 200®), fermented polysaccharide welan gum (K1A96), xanthan gum (Xantural 180®). The names stated in brackets are the trade names by which the materials are known commercially.

A compound selected from among the group comprising crosslinked homo- or copolymers of acrylic acid, gellan gum, propylene glycol alginate, pectin, preferably apple pectin, sodium hyaluronate, xanthan gum, particularly preferably xanthan or a homo- or copolymer of acrylic acid, preferably crosslinked with allylpentaerythritol, is particularly preferably used as a gel-forming agent.

In a particularly preferred embodiment of the present invention, the gel-forming agents used are those which, in addition to the above-stated conditions, also form a gel which encloses air bubbles on extraction from the transdermal system with the necessary minimum quantity of aqueous liquid. The resultant gel is distinguished by a turbid appearance, which provides the potential abuser with an additional optical warning and discourages him/her from administering the gel parenterally.

In general, a quantity of 0.01 to 25 wt. %, preferably of 0.05 to 15 wt. %, particularly preferably of 1 to 10 wt. %, relative to the total weight of the transdermal system, of the stated gel-forming agent is sufficient to prevent abuse.

The gel-forming agent is preferably present in the transdermal system according to the invention in quantities of ≧5 mg, particularly preferably in quantities of ≧10 mg.

If a potential abuser cannot be discouraged from abusive administration by the gelation and optional additional color imparted with component (d), in a preferred embodiment the transdermal system according to the invention ensures that the abusively taken active ingredient does not remain sufficiently long in the abuser's body in order to bring about therein the effects associated with abuse.

This is in particular achieved by also adding an emetic (c) to the transdermal system according to the invention as well as the viscosity-increasing, i.e. gel-forming, agent and optionally the dye (d) having an aversive action.

This emetic is spatially separated from the other components of the transdermal system according to the invention so that it cannot exert any effect on the body when the transdermal system is correctly used. This is ensured by arranging the emetic in a layer which is separated with the assistance of a separation layer which is impermeable to the emetic from the layers containing the other components of the transdermal system according to the invention or by arranging the emetic in microcapsules made from materials which are impermeable to the emetic. Only in the event of inter alia mechanical action by the potential abuser, for example in order to cut up the transdermal system, which usually precedes extraction with an aqueous liquid, are the above-described separation arrangements damaged or cancelled out, such that the emetic is mixed with the remaining components of the transdermal system according to the invention at the latest during extraction. If an abuser takes such a mixture optionally as an extract, the latter triggers the abuse-preventing nausea before the effects associated with abuse are brought about in the body.

Suitable emetics for preventing abuse of an opioid are known to the person skilled in the art and may be present in the transdermal system according to the invention as such or in the form of corresponding derivatives, in particular esters or ethers, or in each case in the form of corresponding physiologically acceptable compounds, in particular in the form of the salts or solvates thereof.

An emetic based on one or more constituents of ipecacuanha (ipecac) root, preferably based on the constituent emetine may preferably be considered in the transdermal system according to the invention, as are, for example, described in “Pharmazeutische Biologie—Drogen und ihre Inhaltsstoffe” by Prof. Dr. Hildebert Wagner, 2nd, revised edition, Gustav Fischer Verlag, Stuttgart, New York, 1982. The corresponding literature description is hereby introduced as a reference and is deemed to be part of the disclosure.

The transdermal system according to the invention may preferably comprise the emetic emetine as component (c), preferably in a quantity of ≧10 mg, particularly preferably of ≧20 mg and very particularly preferably in a quantity of ≧40 mg per transdermal system.

Apomorphine may likewise preferably be used as an emetic for abuse-proofing according to the invention, preferably in a quantity of preferably ≧3 mg, particularly preferably of ≧5 mg and very particularly preferably of ≧7 mg per transdermal system.

If a potential abuser is nevertheless not discouraged from abusive use by the gelation and color imparted by the aversive dye, it is also possible to add to the transdermal system according to the invention as an additional abuse-preventing agent at least one antagonist for the active ingredient with potential for abuse in order to prevent the abusive effects on the abuser.

In this case too, the antagonist should be spatially separated from the other constituents of the transdermal system according to the invention, such that the antagonist cannot exert any action when the transdermal system according to the invention is correctly used. This may, as has as already been mentioned above in connection with the emetic component, be achieved in that the antagonist is separated from the other components of the transdermal system according to the invention by a separation layer impermeable to the antagonist, or in that it is encapsulated in microcapsules, the material of which is impermeable to the antagonist. Only in the event of improper, mechanical handling of the transdermal system according to the invention is the antagonist mixed with the other components, such that, in the event of abusive use, the antagonist prevents the effects otherwise usually brought about by abuse.

Suitable antagonists for preventing abuse of the active ingredients are known per se to the person skilled in the art and may be present in the transdermal system according to the invention as such or in the form of corresponding derivatives, in particular esters or ethers, or in each case in the form of corresponding physiologically acceptable compounds, in particular in the form of the salts or solvates thereof.

If the active ingredient present in the transdermal system is an opioid, the antagonist used is preferably an antagonist selected from the group comprising naloxone, naltrexone, nalmefene, nalid, nalmexone, nalorphine or nalbuphine, in each case optionally in the form of a corresponding physiologically acceptable compound, in particular in the form of a base, a salt or solvate. The corresponding antagonists are preferably used in a quantity of ≧10 mg, particularly preferably in a quantity of 10 to 100 mg, very particularly preferably in a quantity of 10 to 50 mg, relative to the quantity of active ingredient.

If the transdermal system according to the invention comprises a stimulant as active ingredient, the antagonist is preferably a neuroleptic, preferably at least one compound selected from the group comprising haloperidol, promethazine, fluphenazine, perphenazine, levomepromazine, thioridazine, perazine, chlorpromazine, chlorprothixine, zuclopentixol, flupentixol, prothipendyl, zotepine, benperidol, pipamperone, melperone and bromperidol.

The transdermal system according to the invention preferably comprises these antagonists in a conventional therapeutic dose known to the person skilled in the art, particularly preferably in a quantity of twice to four times the conventional dose.

The provision of the transdermal system according to the invention with a gel-forming agent, an aversive dye and an antagonist for preventing abuse may furthermore be supplemented by the transdermal system according to the invention additionally containing an emetic (c) which, together with the antagonist (b), prevents the abusive action on the abuser and is optionally also used instead of the aversively acting dye.

If a potential abuser is not discouraged from abusive administration despite gelation and optionally aversive coloration, it may be appropriate, apart from the already listed components, such as an emetic and optionally an antagonist, to add at least one irritant to the transdermal system according to the invention in addition to or instead of an antagonist. Irritants which may be considered are those substances capable of causing inflammation, fever, burning and/or itching.

Suitable irritants which cause fever and/or inflammation and in particular can be effective against any repetition of the abuse include inter alia lipopolysaccharides and/or microorganisms, such as lactobacilli or Saccharomyces species. These substances are effective not only against parenteral, preferably intravenous abuse, as they may preferably cause inflammation right at the injection site, but also against oral and/or nasal abuse. The latter also applies to irritants which cause burning and/or itching. Appropriate substances and the quantities thereof which are conventionally to be used are known per se to the person skilled in the art and may be identified by simple preliminary testing.

Irritants which cause burning or itching are preferably based on one or more constituents or one or more plant parts of at least one hot substance drug.

Corresponding hot substance drugs are known to the person skilled in the art and are described, for example, in “Pharmazeutische Biologie—Drogen und ihre Inhaltsstoffe” by Prof. Dr. Hildebert Wagner, 2nd, revised edition, Gustav Fischer Verlag, Stuttgart-New York, 1982, pages 82 et seq. The corresponding description is hereby introduced as a reference and is deemed to be part of the disclosure.

One or more constituents of at least one hot substance drug selected from the group comprising Allii sativi bulbus (garlic), Asari rhizoma cum herba (Asarum root and leaves), Calami rhizoma (calamus root), Capsici fructus (capsicum), Capsici fructus acer (cayenne pepper), Curcumae longae rhizoma (turmeric root), Curcumae xanthorrhizae rhizoma (Javanese turmeric root), Galangae rhizoma (galangal root), Myristicae semen (nutmeg), Piperis nigri fructus (pepper), albae semen (white mustard seed), Sinapis nigri semen (black mustard seed), Zedoariae rhizoma (zedoary root) and Zingiberis rhizoma (ginger root), particularly preferably from the group comprising Capsici fructus (capsicum), Capsici fructus acer (cayenne pepper) and Piperis nigri fructus (pepper) may preferably be added as component (a) to the transdermal system according to the invention.

The constituents of the hot substance drugs preferably comprise o-methoxy(methyl)phenol compounds, acid amide compounds, mustard oils or sulfide compounds or compounds derived therefrom.

Particularly preferably, at least one constituent of the hot substance drugs selected from the group comprising myristicin, elemicin, isoeugenol, α-asarone, safrole, gingerols, xanthorrhizol, capsaicinoids, preferably capsaicin, capsaicin derivatives, such as N-vanillyl-9E-octadecenamide, dihydrocapsaicin, nordihydrocapsaicin, homocapsaicin, norcapsaicin and nomorcapsaicin, piperine, preferably trans-piperine, glucosinolates, preferably based on non-volatile mustard oils, particularly preferably based on p-hydroxybenzyl mustard oil, methylmercapto mustard oil or methylsulfonyl mustard oil, and compounds derived from these constituents is suitable as an abuse-preventing further component.

The transdermal system according to the invention may preferably contain the plant parts of the corresponding hot substance drugs in a quantity of 0.01 to 30 wt. %, particularly preferably of 0.1 to 0.5 wt. %, in each case relative to the total weight of the transdermal system according to the invention.

If one or more constituents of corresponding hot substance drugs are used, the quantity thereof in a transdermal system according to the invention preferably amounts to 0.001 to 0.005 wt. %, relative to the total weight of the transdermal system according to the invention.

When irritants are used, it is again necessary for these to be spatially separated in the transdermal system according to the invention from the other components of the transdermal system. As already explained above, this may be achieved in that the layer of the transdermal system containing irritants is provided with a separation layer which is impermeable to the irritant and effects separation from the other components of the transdermal system according to the invention. It is, however, also possible to add the irritant encapsulated in microcapsules, wherein the microcapsules must be impermeable to the irritant. Only in the event of inter alia mechanical action in the course of preparing abusive use are the irritants mixed with the other components of the transdermal system according to the invention, such that the irritants may exert their action.

The spatial arrangement for separating the abuse-preventing components (a) to (d) requires not only that the separating means must be impermeable to the particular abuse-preventing agent, but furthermore that therapeutic action is unaffected when the transdermal system according to the invention is properly used.

The transdermal system according to the invention preferably assumes the form of a patch. It may then be provided as a reservoir or matrix system (Bauer K. H., Frömming K.-H., Führer C., Pharmazeutische Technologie [Pharmaceutical Technology], pages 381-383; Müller R. H., Hildebrand G. E., Pharmazeutische Technologie: Moderne Arzneiformen [Pharmaceutical Technology: Modern Dosage Forms], Chapter 8).

Due to the selection according to the invention of the agent which forms a gel in an aqueous liquid, it is possible to combine the active ingredient with potential for abuse and the gel-forming agent without spatial separation from one another in the patch, without release of the active ingredient being impaired when the patch is properly used.

The gel-forming agent, like the active ingredient, is preferably present in the patch according to the invention in either dissolved or dispersed form. This also applies to the other components for further prevention of abuse, provided that these are present in microcapsules which may optionally dissolve in at least aqueous liquids.

The agent which forms a gel in aqueous liquid is preferably present in the active ingredient-containing matrix layer or in the active ingredient-containing reservoir of the patch or adjacent thereto, in particular if, on contact with an aqueous liquid, it thickens to form a gel from which virtually no active ingredient is extractable. The gel-forming agent may, however, also be present in a further layer of the patch, in particular if, on contact with the aqueous liquid, the gel-forming agent and the active ingredient are extracted and gelation proceeds in the extract.

In accordance with the matrix system, the patch according to the invention may preferably comprise a backing layer, an active ingredient-containing layer and an adhesive layer, wherein the active ingredient-containing layer may simultaneously be the adhesive layer, in which the active ingredient and preferably the gel-forming agent are present dissolved and/or dispersed in a matrix together with the adhesive. The further abuse-preventing components (a) to (d) may be present in encapsulated form in the active ingredient-containing or active ingredient- and adhesive-containing layer or in a separate layer which is segregated from the active ingredient-containing layer with the assistance of a separation layer which is impermeable to these abuse-preventing components (a) to (d). The patch according to the invention preferably additionally also contains a protective layer.

Adhesives which are preferably used for the adhesive layer of the patch according to the invention are pressure-sensitive adhesives. Examples of polymers which are suitable for this purpose are polyacrylates, polyvinyl ethers, polyisobutylene (PIB), styrene/isoprene or butadiene/styrene copolymers or polyisoprene rubbers. Silicone adhesives, such as for example optionally crosslinked polydimethylsiloxanes, are furthermore suitable. Plastics based on esters of glycines, glycerol or polytetraerythritol, or hydrocarbons, such as polyterpenes, are also suitable. Acrylate-based adhesives are produced by polymerisation of acrylates, methacrylates, alkyl acrylates and/or alkyl methacrylates, with optionally further α,β-unsaturated monomers, such as acrylamide, dimethylacrylamide, dimethylaminoethyl acrylate, hydroxyethyl acrylate, hydroxypropyl acrylate, methoxyethyl acrylate, methoxyethyl methacrylate, acrylonitrile and/or vinyl acetate.

The adhesive layer of the patch according to the invention may also contain skin penetration promoters, fillers (such as zinc oxide or silica), crosslinking agents, antioxidants and/or solvents. The thickness of the adhesive layer is preferably 3 to 100 μm.

The backing layer or outer layer of the patch according to the invention is preferably impermeable to and inert towards the active ingredient, adhesive and the abuse-preventing components (a) to (d). The layer is preferably made of polymers, such as polyester, for example polyethylene terephthalate, polyolefins, such as polyethylenes, polypropylenes or polybutylenes, polycarbonates, polyethylene oxides, polyurethanes, polystyrenes, polyamides, polyimides, polyvinyl acetates, polyvinyl chlorides, polyvinylidene chlorides and/or copolymers such as acrylonitrile/butadiene/styrene copolymers optionally containing paper fibres, textile fibres and/or mixtures thereof, which may if necessary be metallised or pigmented. The backing layer or outer layer of the patch may also consist of a combination of metal foil and polymer layer. The thickness of the backing layer is preferably 3 to 100 μm.

The active ingredient-containing matrix layer of the patch according to the invention may contain matrix-forming polymers, skin penetration promoters, solubilising agents, crosslinking agents, stabilizers, emulsifiers, preservatives, thickeners and/or further conventional auxiliaries.

The matrix-forming polymer used is preferably at least one film-forming polymer selected from among the group comprising hydroxypropylcellulose, carboxymethylcellulose, polyethylenes, chlorinated polyethylenes, polypropylenes, polyurethanes, polycarbonates, polyacrylic acid esters, polyacrylates, polymethacrylates, polyvinyl alcohols, polyvinyl chlorides, polyvinylidene chlorides, polyvinylpyrrolidones, polyethylene terephthalates, polytetrafluoroethylenes, ethylene/propylene copolymers, ethylene/ethyl acrylate copolymers, ethylene/vinyl acetate copolymers, ethylene/vinyl alcohol copolymers, ethylene/vinyloxyethanol copolymers, vinyl chloride/vinyl acetate copolymers, vinylpyrrolidone/ethylene/vinyl acetate copolymers, rubbers, rubber-like synthetic homopolymers, copolymers or block polymers, silicones, silicone derivatives, preferably siloxane/methacrylate copolymers, cellulose derivatives, preferably ethylcellulose or cellulose ethers and mixtures thereof. If the active ingredient-containing layer is simultaneously the adhesive layer, it preferably contains, apart from at least one of the enumerated polymers, at least one of the above-listed adhesives.

N-Methyl-2-pyrrolidone, laurylpyrrolidone, triethanolamine, triacetin, diethylene glycol monoethyl ether, derivatives of fatty acids or fatty alcohols may be used as compounds for improving the solubility of the active ingredient.

If the patch according to the invention is produced in accordance with the reservoir system, the reservoir membrane may consist of inert polymers such as for example polyethylenes, polypropylenes, polyvinyl acetates, polyamides, ethylene/vinyl acetate copolymers and/or silicones. The active ingredient and preferably the gel-forming agent may be present in dissolved or dispersed form in the reservoir. The other abuse-preventing components may, as stated above, be spatially separated therefrom.

Stabilizers which may be used for the active ingredient-containing matrix or the active ingredient-containing reservoir are antioxidants, such as vitamin E, butylhydroxytoluene, butylhydroxyanisole, ascorbic acid, ascorbyl palmitate, and/or chelating agents, such as for example disodium ethylenediaminetetraacetic acid, potassium citrate or sodium citrate.

The active ingredient-containing matrix or the active ingredient-containing reservoir may also contain conventional skin penetration promoters.

The patch according to the invention may also contain in one or more layers at least one plasticiser selected from among the group comprising long-chain alcohols, such as dodecanol, undecanol, octanol, esters of carboxylic acids with polyethoxylated alcohols, diesters of aliphatic dicarboxylic acids, such as adipic acid, and medium-chain triglycerides of caprylic acid and/or capric acid, coconut oil, polyhydric alcohols, such as 1,2-propanediol, esters of polyhydric alcohols, such as glycerol with laevulinic acid or caprylic acid, and etherified polyhydric alcohols.

The peelable protective layer of the patch according to the invention may consist of polyethylene, polyester, polyethylene terephthalate, polypropylene, polysiloxane, polyvinyl chloride or polyurethane and optionally of treated paper fibres, such as for example cellophane and optionally comprise a silicone, fluorosilicone or fluorocarbon coating.

Production of the transdermal system, preferably patch, according to the invention, may proceed in accordance with known production processes comprising process steps such as lamination, coextrusion, stamping, delamination, unwinding, cutting, rewinding, assembly or dispensing (Verpackungs-Rundschau 4/2002, 83-84).

### EXAMPLES

### Example 1

a) Production of a Patch Containing Buprenorphine

1139 g of a 48 wt. % polyacrylate solution of a self crosslinking acrylate copolymer prepared from 2-ethylhexyl acrylate, vinyl acetate, acrylic acid (solvent: ethyl acetate:heptane:isopropanol:toluene:acetylacetonate in the ratio 37:26:26:4:1), 100 g of laevulinic acid, 150 g of oleyl acetate, 100 g of polyvinylpyrrolidone, 150 g of ethanol, 200 g of ethyl acetate and 100 g of buprenorphine base are homogenised. The mixture is stirred for approx. two hours and it is visually checked whether all the solids have dissolved. Evaporative loss is checked by reweighing and the loss of solvent is optionally made up by addition of ethyl acetate.

A transparent polyester film 420 mm in width is coated with the above-described mixture in such a manner that the weight per unit area of the dried adhesive layer is 80 g/m². A polyester film rendered detachable by silicone coating serves as the protective layer.

The solvent is removed by drying with hot air which is passed over the solvent-containing web. The heat treatment causes the solvent to evaporate. The adhesive layer is then covered with the 15 μm thick polyester film. Using suitable cutting tools, an area corresponding to the intended quantity of active ingredient is stamped out and the margins remaining between the individual systems are removed.

b) Production of Abuse-Preventing Adhesive Layers
b.1 An abuse-preventing adhesive layer was produced by firstly dissolving 2 g of Carbopol 980 as gel-forming agent in 100 g of ethanol (96%) with stirring and replacing the evaporated ethanol. 10 g, 5 g and 2 g of this 2% Carbopol 980/ethanol solution were respectively stirred into 10 g, 15 g and 18 g of the polyacrylate solution described in 1a) to produce an adhesive mixture and homogeneously distributed therein.
   A water-soluble yellow dye (FD&C Yellow no. 6), which is approved for food use and is physiologically safe, was furthermore added and homogeneously distributed.
   Using an Erichsen Coatmaster 509/MC-1 film coater, 20 g of the above-listed adhesive mixture containing Carbopol and dye were applied onto a siliconized polyester film (Hostaphan film RNT 36) using a 120 μm blade spacing Application speed was 5 mm/sec. After a drying time of at least 2 hours, a siliconized polyester film was laminated as a protective layer onto the uncoated side of the adhesive layer. 7×7 cm squares were then cut from the abuse-complicating adhesive layers laminated on both sides.
   After removal of the siliconized protective layer, each of the adhesive layers provided with a different concentration of Carbopol and with dye was joined to the exposed adhesive layer of the buprenorphine-containing d obtained according to 1a).
b.2 Production of further abuse-preventing adhesive layers
   Xanthan as the gel-forming compound was sieved through a 50 μm sieve and the fines were further used.
   1 g, 2 g and 3 g of xanthan were in each case suspended in 3 g of ethanol (96%) and the suspension was homogeneously distributed in respectively 19 g, 18 g and 17 g of the polyacrylate solution described in 1a) to produce an adhesive mixture. 3 g of ethanol were stripped out from each mixture using a Rotavapor rotary evaporator and beta-carotene was additionally introduced into the mixture as a dye component. Each resultant mixture was in each case applied as an adhesive layer onto a siliconised polyester film (Hostaphan film RNT 36) with the assistance of a 120 μm blade spacing on an Erichsen Coatmaster 509/MC-1 film coater. Application speed was 5 mm/sec. After a drying time of at least 2 hours, a siliconized polyester film was again laminated as a protective film onto the uncoated side of the adhesive layer. 7×7 cm squares were cut out. After removal of the protective film, each of adhesive layers provided with a different concentration of xanthan and with dye was joined to the likewise exposed adhesive layer of the buprenorphine-containing patch obtained according to 1a).

c) Testing of Abuse-Complicating/-Preventing Action
After removal of the protective layer from the abuse-complicating adhesive layer, the patches obtained according to b.1 and b.2 provided with an abuse-complicating adhesive layer were brought into contact with 5 ml of water. A red or yellowish-red colored gel layer formed on each patch, wherein, even after a contact time of 5 hours, no buprenorphine could be detected in the remaining water not absorbed by the gel layer.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. An abuse-resistant transdermal system comprising:
at least one pharmaceutically active ingredient with potential for abuse;
at least one gel-forming agent in a quantity such that it forms a gel with a minimum quantity of an aqueous liquid, and
at least one agent which complicates or prevents abuse selected from the group consisting of emetics and dyes which in aqueous solution impart an intense color.

2. A transdermal system according to claim 1, further comprising at least one further agent which complicates or prevents abuse selected from irritants and antagonists for the pharmaceutically active ingredient with potential for abuse.

3. A transdermal system according to claim 1, wherein said gel cannot be administered parenterally.

4. A transdermal system according to claim 1, wherein the at least one pharmaceutically active ingredient is present in an active ingredient-containing zone of the transdermal system, and the gel-forming agent is present in a quantity of from 0.01 to 25 wt. %, relative to the total weight of the active ingredient-containing zone.

5. A transdermal system according to claim 1, wherein the at least one gel-forming agent is/are selected from the group consisting of carbomers, locust bean flour, sodium carboxymethylcellulose, sodium alginate, guar flour, iota-carrageenan, karaya gum, gellan gum, tara stone flour, propylene glycol alginate, pectins, sucrose acetate isobutyrate, sodium hyaluronate, fermented polysaccharides and xanthans.

6. A transdermal system according to claim 5, wherein the at least one gel-forming agent is/are selected from the group consisting of crosslinked homo- or copolymers of acrylic acid, gellan gum, propylene glycol alginate, apple pectin, sodium hyaluronate, and xanthan gum.

7. A transdermal system according to claim 6, wherein the at least one gel-forming agent is an apple pectin or a xanthan gum.

8. A transdermal system according to claim 1, wherein said at least one pharmaceutically active ingredient with potential for abuse is selected from the group consisting of opioids, tranquilizers, stimulants and narcotics.

9. A transdermal system according to claim 8, wherein said pharmaceutically active ingredient with potential for abuse is an opioid selected from the group consisting of morphine, oxycodone, buprenorphine, sulfentanil, hydromorphone, carfentanil, lofentanil, and fentanyl.

10. A transdermal system according to claim 1, wherein said at least one agent which complicates or prevents abuse comprises an emetic selected from the group consisting of ipecacuanha root, emetine, and apomorphine.

11. A transdermal system according to claim 1, wherein said at least one agent which complicates or prevents abuse comprises a dye which imparts an intense color in aqueous solution.

12. A transdermal system according to claim 1, wherein said at least one agent which complicates or prevents abuse comprises an emetic and an aversive dye.

13. A transdermal system according to claim 2, wherein said transdermal system comprises at least one opioid antagonist, said at least one opioid antagonist being selected from the group consisting of naloxone, naltrexone, nalmephine, nalid, nalmexone, nalorphine, nalbuphine, and transdermally administrable physiologically acceptable salts, esters or ethers of any of the foregoing.

14. A transdermal system according to claim 2, wherein said transdermal system comprises at least one irritant selected from the group consisting of inflammation-causing substances and fever-causing substances.

15. A transdermal system according to claim 14, wherein said at least one irritant is a fever-causing substance selected from the group consisting of lipopolysaccharides and fever-causing microorganisms.

16. A transdermal system according to claim 15, wherein said fever-causing substance is a fever-causing microorganism selected from the group consisting of lactobacilli and Saccharomyces.

17. A transdermal system according to claim 14, wherein said irritant comprises a plant substance which produces a hot sensation selected from the group consisting of Capsici fructus, Capsici fructus acer, and Piperis nigri fructus.

18. A transdermal system according to claim 1, wherein said at least one agent which complicates or prevents abuse is spatially separated from other components of the transdermal system.

19. A transdermal system according to claim 18, wherein the spatial separation is effected by encapsulation of the emetic or aversive dye in microcapsules made from a material impermeable to said emetic or aversive dye.

20. A transdermal system according to claim 2, wherein said antagonist or irritant is spatially separated from other components of the transdermal system.

21. A transdermal system according to claim 20, wherein the spatial separation is effected by encapsulation of the antagonist or irritant in microcapsules made from a material impermeable to said emetic or aversive dye.

22. A transdermal system according to claim 1, wherein the at least one agent which complicates or prevents abuse is separated from other components of the transdermal system by a separation layer impermeable to the agents which complicate or prevent abuse.

23. A transdermal system according to claim 1, wherein the gel-forming agent is present in dissolved or dispersed form.

24. A transdermal system according to claim 1, wherein the transdermal system is a transdermal patch.

25. A transdermal system according to claim 24, wherein said patch is a reservoir patch which contains the pharmaceutically active ingredient in a reservoir.

26. A transdermal system according to claim 25, wherein the gel-forming agent is present in the active ingredient-containing reservoir of the reservoir patch and each of the agents which complicate or prevent abuse is encapsulated in microcapsules impermeable to the encapsulated agent and also disposed the reservoir.

27. A transdermal system according to claim 24, wherein said transdermal patch is matrix patch which contains the pharmaceutically active ingredient in a matrix.

28. A transdermal system according to claim 27, wherein the gel-forming agent is present in the active ingredient-containing matrix of the matrix patch in dissolved or dispersed form, and each of the agents which complicate or prevent abuse is encapsulated in microcapsules impermeable to the encapsulated agent and also disposed the matrix.
